(19) United States
(12) Patent Application Publication
(10) Pub. No.: US 2016/0082156 A1
(43) Pub. Date: Mar. 24, 2016

(54) OSTEOINDUCTIVE SUBSTRATES AND METHODS OF MAKING THE SAME

(71) Applicants: Christopher G. WILSON, Durham, NC (US); Eric J. Vanderploeg, Durham, NC (US); Howard Seeherman, Durham, NC (US); Zachary Decker, Durham, NC (US); Eric Schmidt, Durham, NC (US); Bethany Moore, Durham, NC (US); Alexander Heubeck, Durham, NC (US)

(72) Inventors: Christopher G. WILSON, Durham, NC (US); Eric J. Vanderploeg, Durham, NC (US); Howard Seeherman, Durham, NC (US); Zachary Decker, Durham, NC (US); Eric Schmidt, Durham, NC (US); Bethany Moore, Durham, NC (US); Alexander Heubeck, Durham, NC (US)

(21) Appl. No.: 14/839,786

(22) Filed: Aug. 28, 2015

Related U.S. Application Data
(60) Provisional application No. 62/043,356, filed on Aug. 28, 2014.
(60) Provisional application No. 62/155,835, filed on May 1, 2015.

(51) Int. Cl. A61L 27/44; A61F 2/28

(52) U.S. Cl. CPC A61L 27/44; A61F 2/28; A61L 2430/24; A61F 2002/2835; A61F 2002/2817

(57) ABSTRACT

Systems and methods for preparing osteoinductive synthetic bone grafts are provided in which a porous ceramic granule is loaded with an osteoinductive material, and then placed in contact with a biocompatible matrix material.

### CROSS-REFERENCE TO RELATED APPLICATIONS

This application claims the benefit of priority under 35 U.S.C. §119(e) to U.S. provisional patent application No. 62/043,356 filed Aug. 28, 2014 by Vanderploeg, et al. and to U.S. provisional application No. 62/155,835 filed May 1, 2015 by Decker, et al. Each of the foregoing applications is incorporated by reference in its entirety herein.

### FIELD OF THE INVENTION

This application relates to medical devices and biologic therapies, and more particularly to substrates for bone repair which include protein-loaded matrices.

### BACKGROUND

Bone grafts are used in roughly two million orthopedic procedures each year, and generally take one of three forms. Autografts, which typically consist of bone harvested from one site in a patient to be grafted to another site in the same patient, are the benchmark for bone grafting materials, inasmuch as these materials are simultaneously osteoconductive (it serves as a scaffold for new bone growth), osteoinductive (promotes the development of osteoblasts) and osteogenic (contains osteoblasts which form new bone). However, limitations on the supply of autografts have necessitated the use of cadaver-derived allografts. These materials are less ideal than autografts, however, as allografts may trigger host-graft immune responses or may transmit infectious or prion diseases, and are often sterilized or treated to remove cells, eliminating their osteogenicity.

The shortcomings of human-derived bone graft materials have contributed to a growing interest in synthetic bone graft materials. Synthetic grafts typically comprise calcium ceramics and/or cements delivered in the form of a paste or a putty. These materials are osteoconductive, but not osteoinductive or osteogenic. To improve their efficacy, synthetic calcium-containing materials have been loaded with osteoinductive materials, particularly bone morphogenetic proteins (BMPs), such as BMP-2, BMP-7, or other growth factors such as fibroblast growth factor (FGF), insulin-like growth factor (IGF), platelet-derived growth factor (PDGF), and/or transforming growth factor beta (TGF-β). However, significant technical challenges have prevented the efficient incorporation of osteoinductive materials into synthetic bone graft substitutes which, in turn, has limited the development of high-quality osteoinductive synthetic bone graft materials.

### SUMMARY OF THE INVENTION

The present invention addresses the shortcomings of current-generation synthetic bone grafts by providing graft materials with improved loading of osteoinductive materials, as well as methods of making and using the same. In one aspect, the present invention relates to a system for forming a composite osteoinductive scaffold that includes an osteoinductive material (which material is generally, but not necessarily, a protein or peptide and is, in the exemplary embodiments described here, referred to interchangeably as an “osteoinductive protein”), at least one of a calcium ceramic granule and a flowable biocompatible matrix material, and an apparatus defining at least one chamber and at least one inlet for introducing the protein, granule and/or matrix material into the chamber. In various cases, the osteoinductive material is in aqueous solution, and/or the apparatus includes a static mixing element (e.g. within or fluidly connected to the chamber(s)). In some cases, one or more of the granules, the osteoinductive material, and/or the flowable biocompatible matrix material includes or is integrated into the scaffold alongside a porogen, which porogen is optionally a removable particle having an average size similar to an average size of the granule that is leachable, collapsible, dissolvable or otherwise degradable. The matrix is optionally selected from the group consisting of hyaluronic acid (HA), modified HA, collagen, gelatin, fibrin, chitosan, alginate, agarose, a self-assembling peptide, whole blood, platelet-rich plasma, bone marrow aspirate, polyethylene glycol (PEG), a derivative of PEG, poly(lactide-co-glycolide), poly(caprolactone), poly(lactic acid), poly(glycolic acid), a poloxamer, and copolymers or combinations thereof. Where a granule is used, it is generally porous and may include a material selected from the group comprising monocalcium phosphate monohydrate, dicalcium phosphate, dicalcium phosphate dehydrate, octocalcium phosphate, precipitated hydroxyapatite, precipitated amorphous calcium phosphate, monocalcium phosphate, alpha-tricalcium phosphate (α-TCP), beta-tricalcium phosphate (β-TCP), sintered hydroxyapatite, oxyapatite, tetracalcium phosphate, hydroxyapatite, calcium-deficient hydroxyapatite, and combinations thereof. The osteoinductive material is, optionally, selected from the group consisting of bone morphogenetic protein 2 (BMP-2), BMP-3, BMP-4, BMP-5, BMP-6, BMP-7, BMP-9, a designer BMP, fibroblast growth factor, insulin-like growth factor, platelet-derived growth factor, transforming growth factor beta (TGF-β), and combinations thereof. The apparatus, meanwhile, optionally includes a structure to improve mixing of the elements incorporated into the scaffold, such as a static mixing element within or connectable-to the chamber, and/or a fenestrated needle insertable into the chamber. Various embodiments of the system may be used, in some instances, to perform the methods and/or form medical implants as described in greater detail below.

In another aspect, the present invention relates to a method of preparing a synthetic graft material (optionally, but not necessarily using one of the systems described above) that includes loading or associating a calcium ceramic granule with an osteoinductive material, for instance by contacting the granule with a solution comprising the osteoinductive material, which solution optionally includes a reagent that facilitates the subsequent formation of a biocompatible matrix in association with the granules, such as a gelling reagent, gelling catalyst, and/or a cross-linking agent. The method may also include embedding the protein-loaded calcium ceramic granule in a biocompatible matrix, which can be flowed over the protein-loaded granules (e.g. by flowing a flowable matrix material into a chamber containing the protein-loaded granules). In various embodiments, the osteoinductive material is BMP-2, BMP-4, BMP-6, BMP-7, or a designer BMP. The calcium ceramic granule includes, variously, calcium sulfates and calcium phosphates such as hydroxyapatite, tri-calcium phosphate, calcium-deficient hydroxyapatite, or combinations thereof, while the biocompatible matrix material is, in various embodiments, hyaluronic acid (HA), and functionalized or modified versions thereof, collagen, whether animal or recombinant human, gelatin (animal or recombinant human), fibrin, chitosan, alginate, agarose, self-assembling peptides, whole blood, platelet-rich plasma, bone marrow aspirate, polyethylene glycol (PEG) and derivatives thereof, functionalized or otherwise cross-linkable synthetic biocompatible polymers including poly(lactide-co-glycolide), poly(caprolactone), poly(lactic acid), poly(glycolic acid), poloxamers and other thermosensitive or reverse-thermosensitive polymers known in the art, and copolymers or admixtures of any one or more of the foregoing. The biocompatible matrix material is, in some cases, reactive, and can be triggered to undergo one or more of a polymerization reaction and a cross-linking reaction to form a gel or other polymer mass; when this is the case, the reaction(s) optionally take between 30 seconds and 5 minutes, and the matrix material can optionally be flowed over and/or mixed with the granules during this interval, thereby facilitating the formation of more homogeneous implants.

In another aspect, the present invention relates to an implant formed using the systems and/or methods described above, which implant includes a biocompatible matrix, an osteoinductive material associated with an interior surface (e.g. a pore surface) of a calcium ceramic granule, which calcium ceramic granule is, in turn, associated with the matrix. In preferred cases, the implant is substantially uniform, i.e. a concentration of the osteoinductive material and one or more of the calcium ceramic granule and the biocompatible matrix material is substantially constant along at least one physical dimension of the implant.

In yet another aspect, the invention relates to a method of treating a patient, comprising delivering a composition including calcium ceramic granules loaded or associated with an osteoinductive material, the granules embedded in a biocompatible matrix. In various embodiments, the osteoinductive material is BMP-2, BMP-4, BMP-6, BMP-7, or a designer BMP. The calcium ceramic granule includes, variously, calcium sulfates and calcium phosphates such as hydroxyapatite, tri-calcium phosphate, calcium-deficient hydroxyapatite, or combinations thereof, while the biocompatible matrix is, in various embodiments, hyaluronic acid (HA), and functionalized or modified versions thereof, collagen, whether animal or recombinant human, gelatin (animal or recombinant human), fibrin, chitosan, alginate, agarose, self-assembling peptides, whole blood, platelet-rich plasma, bone marrow aspirate, polyethylene glycol (PEG) and derivatives thereof, functionalized or otherwise cross-linkable synthetic biocompatible polymers including poly(lactide-co-glycolide), poly(caprolactone), poly(lactic acid), poly(glycolic acid), poloxamers and other thermosensitive or reverse-thermosensitive polymers known in the art, and copolymers or admixtures of any one or more of the foregoing.

And in yet another aspect, the present invention relates to a kit for treating a patient with an osteoinductive material. The kit includes, generally, a calcium ceramic granule, an osteoinductive material, and a biocompatible scaffold material, as well as mechanical tools for combining them to form an osteoinductive synthetic bone graft. In some cases, the kit includes a vessel that includes a chamber for holding the granules as well as inlets and outlets via which fluids can be supplied to and/or withdrawn from the chamber. In one scheme, the granules are loaded with the osteoinductive material by flowing a liquid comprising the osteoinductive material through the inlet and contacting the granules therewith; thereafter, the granules are mixed with or otherwise placed in contact with the biocompatible matrix material.

### BRIEF DESCRIPTION OF THE DRAWINGS

Certain embodiments of the present invention are illustrated by the accompanying figures. It will be understood that the figures are not necessarily to scale and that details not necessary for an understanding of the invention or that render other details difficult to perceive may be omitted. It will be understood that the invention is not necessarily limited to the particular embodiments illustrated herein.

FIG. 1A-B shows two constructs of the invention with different macroporosities.

FIG. 2A-C shows several steps in an exemplary method of preparing a synthetic, osteoinductive bone graft material.

FIG. 3A shows an exemplary mixing apparatus comprising a fenestrated needle.

FIG. 3B illustrates the direction of fluid flow in an exemplary mixing chamber. FIGS. 3C-N show cross-sectional views of scaffolds made using no needle (C through H) and a centrally-positioned fenestrated needle (I through N) to apply solutions of osteoinductive factors (here, BMP2 in cyan) and biocompatible matrix to the granules in otherwise similar mixing apparatuses. These figures illustrate the improved distribution of osteoinductive materials, granules and matrix materials achieved using a fenestrated needle to apply the solutions in comparison to the apparatus without an embedded fenestrated needle.

FIGS. 4A-B show the cylindrical implant after mixing and trimming the ends (2A) and formed within the syringe after mixing (2B).

FIG. 5 shows a slice of the cylindrical implant on a rheometer prior to a compression test.

FIG. 6 is a graph comparing the phase difference between the shear storage (G′) and shear loss (G″) moduli for various hydrogel compositions.

FIG. 7 is a graph showing the time required for each hydrogel composition to complete the cross-linking reaction.

FIG. 8 is a graph showing the maximum stiffness of each hydrogel composition.

FIG. 9 shows various connector designs for attaching two mixing syringes.

FIGS. 10A-D show variations of the internal mixing structure used to test mixing potential. Static mixer designs include a hollow tube (10A), semi-sphere (10B), single crossbar (10C) and double crossbar (10D).

FIGS. 11A-B show a machine milled prototype of a connecter made with transparent plastic (11A) to allow visualization of the mixing procedure (11B).

FIG. 12 shows the waste material that accumulates within a connector that includes a semi-sphere static mixer.

FIGS. 13A-B show a 3-D printed connector that includes a single crossbar static mixer (13A) connected to two mixing syringes (13B).

FIGS. 14A-B are graphs showing the average values of the elastic moduli (14A) and density (14B) of each 5 mm section for each hydrogel composition tested.

FIG. 15 is a graph showing the variability in mechanical properties of hydrogel compositions composed of different granule concentrations.

FIG. 16 is a graph showing the deviation in mechanical properties between slices of hydrogel compositions with the same granule composition.

FIG. 17 is a graph showing the normalized intensity values of fluorescently tagged albumin between slices of each hydrogel composition.

FIG. 18 is a graph showing the deviation of fluorophore tagged albumin fluorescence for each hydrogel composition.

FIG. 19 is a graph showing the normalized intensity values of BMP-2 tagged with AF488 within four slices of a hydrogel composition that includes 20% or 30% granules.

FIGS. 20A-B are confocal images of a hydrogel composition containing 30% granules by volume with BMP-2 diluted in BMP buffer.

FIGS. 21A-B are graphs showing the average intensities of fluorescence emission from AF488 tagged BMP-2.

### DETAILED DESCRIPTION

### Osteoinductive Compositions

Implants (also referred to as “constructs”) according to the various embodiments of the present invention generally include three components: an osteoconductive material, such as a calcium ceramic or other solid mineral body, an osteoinductive material such as a bone morphogenetic protein, and a flowable biocompatible matrix material that reacts to form a gel or other mass. As used herein, osteoconductive materials refer to any material which facilitates the ingrowth of osteoblastic cells including osteoblasts, pre-osteoblasts, osteoprogenitor cells, mesenchymal stem cells and other cells which are capable of differentiating into or otherwise promoting the development of cells that synthesize and/or maintain skeletal tissue. In preferred embodiments of the present invention, the osteoconductive material is a porous granule comprising an osteoconductive calcium phosphate ceramic that is adapted to provide sustained release of an osteoinductive substance that is loaded onto the granule. In some cases, the granule includes both micro- and macro-pores that define surfaces on which the osteoinductive substance can adhere or otherwise associate. Both micro-pores and macro-pores increase the total surface area to which the osteoinductive substance can adhere, but only the macro-pores permit infiltration by cells. Thus, osteoinductive substance within the micro-pores becomes available only gradually, as the granule is degraded by cells infiltrating the macro-pores.

The granules can be made of any suitable osteoconductive material having a composition and architecture appropriate to allow an implant of the invention to remain in place and to release osteoinductive material over time intervals optimal for the formation and knitting of bone (e.g. days, weeks, or months). While these characteristics may vary between applications, the granules generally include, without limitation, monocalcium phosphate monohydrate, dicalcium phosphate, dicalcium phosphate dehydrate, octocalcium phosphate, precipitated hydroxyapatite, precipitated amorphous calcium phosphate, monocalcium phosphate, alpha-tricalcium phosphate (α-TCP), beta-tricalcium phosphate (β-TCP), sintered hydroxyapatite, oxyapatite, tetracalcium phosphate, hydroxyapatite, calcium-deficient hydroxyapatite, and combinations thereof.

With respect to granule architecture, in preferred embodiments, the granules are characterized by (a) surface area and (b) porosity which, again, are selected to allow an implant of the invention to remain in place and to release osteoinductive material over time intervals optimal for the formation and knitting of bone (e.g. days, weeks, or months). Porosity has two components: microporosity and macroporosity, which can be selected to achieve desired granule residence times or kinetics of release of osteoinductive materials. Microporosity generally refers to the existence of pores with a relatively narrow average diameter that is nonetheless large enough to permit infiltration of fluids such as BMP-loaded solutions into micropores without immediately contacting a surface of the micropore (i.e. sufficiently large to permit fluid access without excessive surface tension). Macroporosity, with respect to granules, generally refers to the existence of pores sized to permit infiltration by cells.

Osteoinductive materials generally include peptide and non-peptide growth factors that stimulate the generation of, or increase the activity of, osteoblasts and/or inhibit the activity or generation of osteoclasts. In some embodiments, the osteoinductive material is a member of the transforming growth factor beta (TGF-β) superfamily such as TGF-β. More preferably, the osteoinductive material is a bone morphogenetic protein (BMP) such as BMP-2, BMP-3, BMP-4, BMP-5, BMP-6, BMP-7, BMP-9, or a designer BMP such as the BMP-GER or BMP-GER-NR chimeric BMPs described in U.S. Pre-grant application publication no. US 20120046227 A1 by Berasi et al. entitled “Designer Osteogenic Proteins,” the entire disclosure of which is hereby incorporated by reference for all purposes. In other embodiments, the osteoinductive material is a fibroblast growth factor, insulin-like growth factor, platelet-derived growth factor, a small molecule, a nucleotide, a lipid, or a combination of one or more of the factors listed herein.

Various embodiments of the invention utilize a biocompatible matrix, which can be any suitable biocompatible material which preferably (a) when used in concert with the granules, exhibits sufficient rigidity and/or column strength to withstand the loads placed upon it when implanted, (b) which does not cause excessive inflammation (i.e. inflammation sufficient to inhibit or prevent the formation of new bone or the knitting of a broken bone), inhibit the proliferation of osteoblasts, or otherwise interfere with the activity of the granules and/or the osteoinductive material, and (c) has sufficient cohesion over an appropriate interval to permit the deposition of new bone. In addition, the biocompatible matrix is optionally degradable and/or osteoconductive. The biocompatible matrix is, in preferred embodiments, made from a flowable precursor material that reacts to form a gel or other solid mass, for example by polymerizing and/or cross-linking in the presence of the granules. In various embodiments, the matrix includes hyaluronic acid (HA), and functionalized or modified versions thereof, collagen, whether animal or recombinant human, gelatin (animal or recombinant human), fibrin, chitosan, alginate, agarose, self-assembling peptides, whole blood, platelet-rich plasma, bone marrow aspirate, polyethylene glycol (PEG) and derivatives thereof, functionalized or otherwise cross-linkable synthetic biocompatible polymers including poly(lactide-co-glycolide), poly(caprolactone), poly(lactic acid), poly(glycolic acid), poloxamers and other thermosensitive or reverse-thermosensitive polymers known in the art, and copolymers or admixtures of any one or more of the foregoing. The reaction process by which the matrix materials form a gel or other mass is preferably a short, but not instant process, that takes, for instance, 30 seconds, 1 minute, 5 minutes, up to 10 minutes, to permit the material to be flowed over and/or mixed with the granules and to form a relatively homogeneous mixture, which will give rise to a compositionally (and thus mechanically) homogeneous implant.

In some cases, the matrix material requires one or more of a catalyst and a co-reactant in order to react to form the gel or mass. The catalyst or co-reactant may be provided simultaneously with the matrix material or, in some cases, may be provided prior to the introduction of the matrix material. In one example, a reagent necessary for the selected matrix material to undergo an enzymatically-catalyzed cross-linking reaction, (hydrogen peroxide) was included in the solution of osteoinductive material applied to the granules. Thus, crosslinking began when the polymer solution came in contact with the granules. Other crosslinking or polymerizing agents, such as hydrogen peroxide, a photoinitiator, or a divalent cation may also be added to the granules before the addition of the matrix material.

Implants or constructs of the invention, which include the osteoinductive materials, granules and biocompatible matrices as described above, also have characteristics which are tailored to the facilitation of bone growth and knitting, which include (a) kinetics of release of osteoinductive materials that are appropriate for the application, (b) residence time appropriate to facilitate but not interfere with new bone formation, (c) macroporosity that permits the infiltration of cells and tissues, including new vascular tissue that accompanies the formation of new bone, and (d) sufficient rigidity/or and compression resistance to withstand loads applied to the implant.

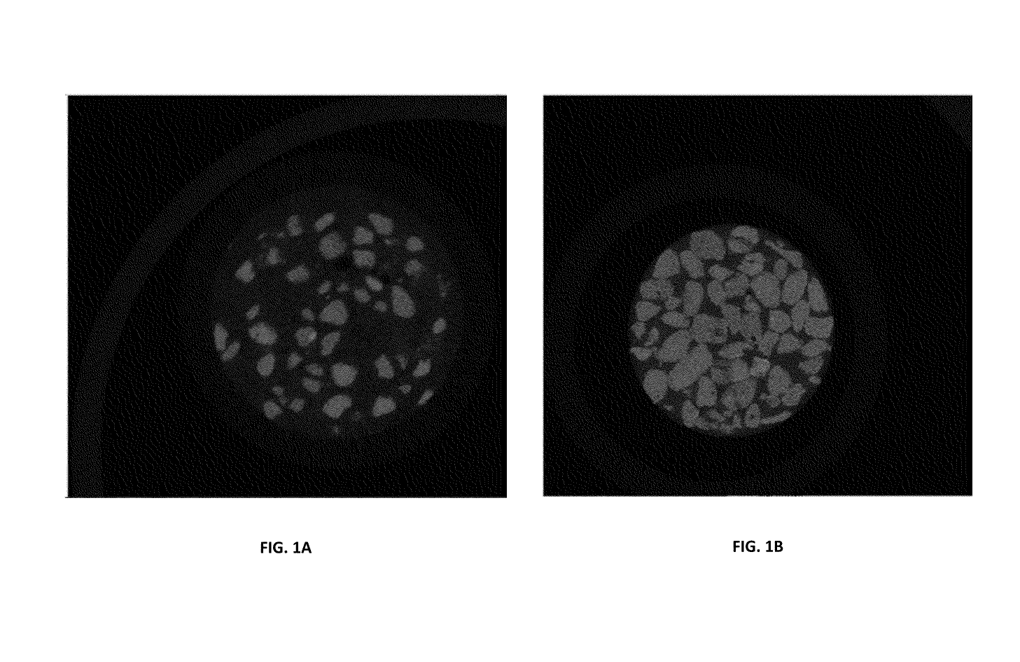

As to macroporosity, FIG. 1 shows constructs of the invention with relatively high (FIG. 1A) and relatively low (FIG. 1B) porosity. The constructs shown in cross section in FIG. 1 are compositionally similar to one another, but the construct of FIG. 1A incorporated sucrose crystals sized as a porogen, while the construct of FIG. 1B did not. While not wishing to be bound by any theory, it is believed that, without the addition of porogens, the porosity of the construct will vary with granule size: the larger the size of the ceramic granules used, the larger the spaces between them. However, when the ceramic granules are in the 300 to 500 micron range, as in certain embodiments of the invention, and as illustrated in FIG. 1B, the pores between the granules will typically fall below the ideal porosity (also 300 to 500 microns) without the addition of a porogen.

### Loading Procedures

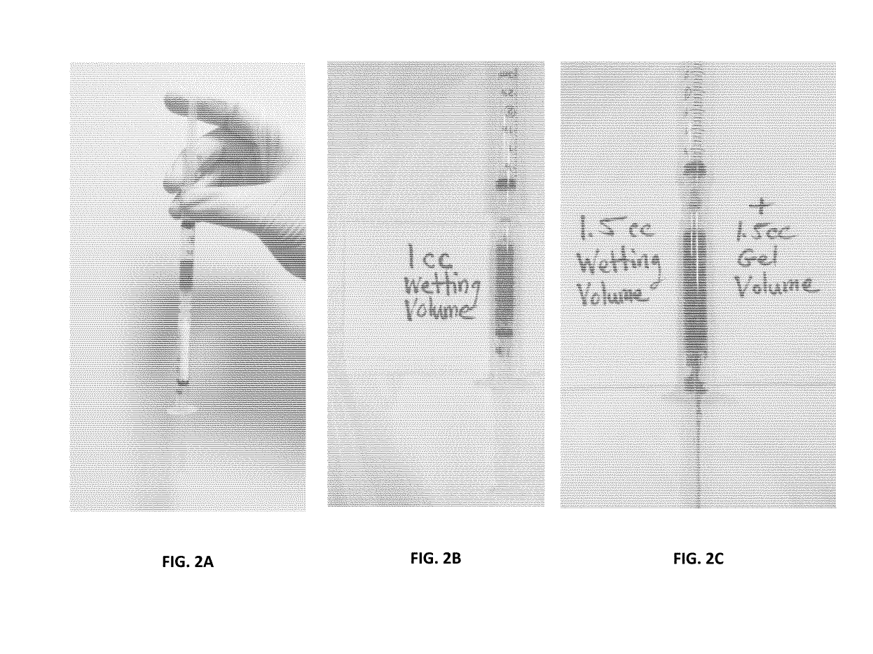

The synthetic bone graft materials of the present invention are generally prepared by the sequential combination of granules, osteoinductive material, and biocompatible matrix material. FIG. 2A-C depict an exemplary two-step process for preparing a synthetic bone graft. First, as shown in FIG. 2A-B, an osteoinductive material, such as a BMP, is applied to the granules, for instance by flowing a solution containing the osteoinductive material over the granules to permit the material to adhere to various surfaces within the granules, including the internal pore surfaces (if any). The volume of solution applied to the granules is, in preferred embodiments, sufficient to fully wet the granules, thereby ensuring that all surfaces (including internal pore surfaces) are incubated with the osteoinductive material. The incubation of the granules may be over a variety of intervals, temperatures, pressures (as may be necessary to facilitate complete infiltration of micropores) or may otherwise be manipulated in any suitable way to tailor the combination of the osteoinductive material and the granules. Infiltration of fluids into the granules is optionally facilitated by the inclusion of one or more surfactants.

Following the loading step, the granules are embedded into the biocompatible matrix. In some cases, as shown in FIG. 2C, a formulation that generates a matrix, such as a cross-linkable prepolymer, is applied to the granules and reacted to form the matrix. A porogen is preferably added to the formulation such that the resulting construct has a suitable macroporosity (e.g. between 300 and 500 micron pores). Any porogen may be used, though in preferred embodiments the porogen is biocompatible, is provided as particles sized similarly to the ceramic granules used in the construct, and has a density that is greater than, or at least not substantially less than, that of the matrix-generating formulation so that it is not displaced or diluted during the formation of the matrix. Where a leachable particle is used, it is preferably relatively insoluble in the formulation, so that it remains in the solid phase while the biocompatible matrix is formed. In some embodiments, microspheres which are configured to collapse or dissolve in response to the application of external energy, such as ultrasound or UV light, are used, while in other embodiments, a thermosensitive porogen particle, such as a thermosensitive (or reverse-thermosensitive) polymer bead, is used as a porogen.

Following the formation of the construct, the porogen may degrade or be removed rapidly, or may remain in place even after the construct is implanted into a patient. In preferred embodiments, the porogen remains intact for hours or days, but less than one week.

In addition to, or in lieu of, the polymer compositions described above, the biocompatible matrix may comprise other materials useful in the treatment of bone, such as acrylate polymer materials (for instance polymethylmethacrylate), demineralized bone, calcium phosphate putty, and the like.

In some cases, the loading of the granules and/or their placement in the biocompatible matrix is done in suite, by an end user. Such in suite loading is facilitated by a kit that includes, in an exemplary embodiment, a vessel for holding the granules and into which the osteoinductive material and/or the biocompatible matrix can be flowed. In preferred embodiments, the vessel includes an inlet, an outlet, and a space for holding the plurality of granules. One or more of the inlet and the outlet are connectable to a fluid source, for instance by means of a male or female luer tip. The kit also optionally includes one or more of a filter for limiting the incorporation of aggregates of the osteoinductive material and/or for preventing the escape of granules and a static mixer to improve mixing of materials flowed therethrough. Additionally, one or more of the osteoinductive material and the biocompatible matrix material can be provided in liquid form, for instance in a pre-loaded syringe, or in reconstitutable form (e.g. in a vial in lyophilized or freeze-dried form together with a diluent for reconstitution). Where a porogen is used, it can be supplied separately, for mixing with the biocompatible matrix immediately before its application to the loaded granules, or it can be mixed in with one or more of the granules, the osteoinductive material (e.g. in solution therewith) and/or the biocompatible matrix, if stable therewithin. For instance, where a leachable porogen particle (such as an inorganic salt crystal) is used, it can be provided separately and then added to an incubation of the granules with the osteoinductive material and/or the biocompatible matrix material, or it may be provided together with, for instance, a lyophilized biocompatible matrix material that is wetted with a diluent (e.g. water) prior to the application of the matrix-material to the granules; the leachable porogen is provided in the form of particles or grains that are roughly the same size and is relatively insoluble in the diluent

To use a kit of the invention, a user first connects the vessel containing the granules to a source of a first solution containing the osteoinductive material, flows the first solution into the vessel and over the granules. Next, the user disconnects the source of the first solution and connects a source of a second solution containing a biocompatible matrix material. Following formation of the matrix, the graft is removed and optionally prepared for implantation into a patient, for instance by trimming and/or loading into an implant.

Certain principles of the present invention are illustrated by the following non-limiting examples:

### Example 1

### Testing of Hydrogel Characteristics

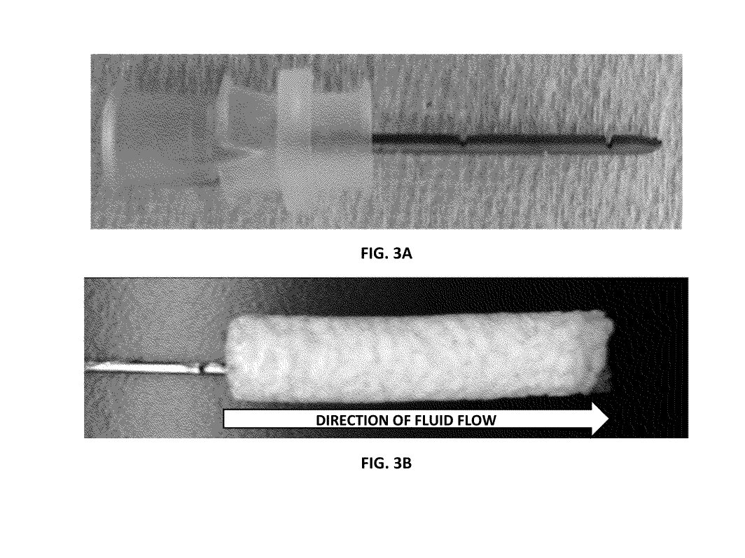

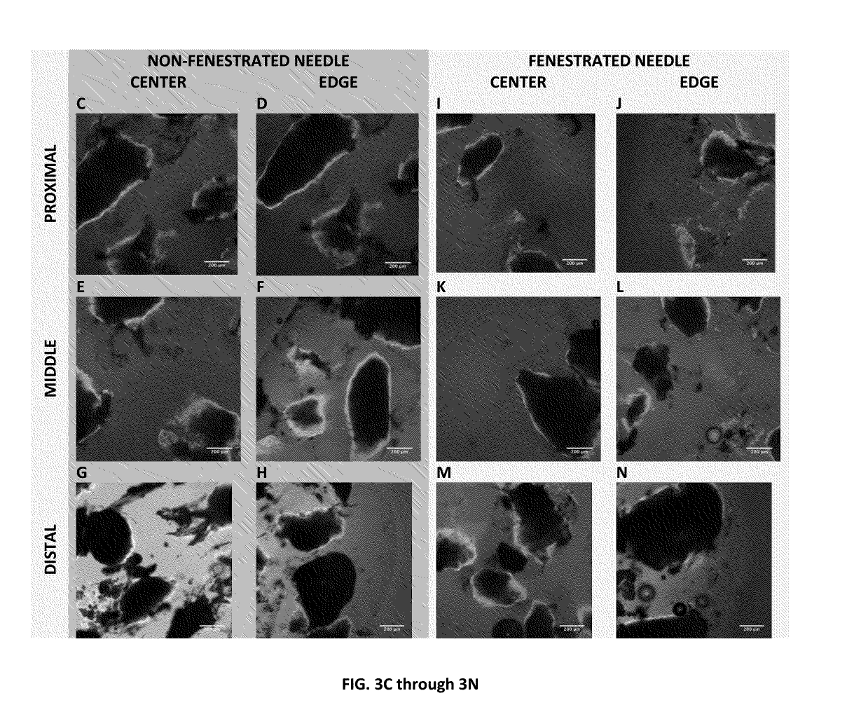

In some cases, the devices, systems and methods of the present disclosure may be used to uniformly load osteoinductive materials onto calcium phosphate granules within a hydrogel scaffold. One potential hydrogel material is tyramine-substituted hyaluronic acid (HA). To understand the flow characteristics of such hydrogels, oscillation and flow testing on hydrogels with various substitutions of the cross-linking active tyramine base (1%, 3% and 5%) at specific concentrations (5 mg/ml or 10 mg/ml) as shown in FIG. 3 were analyzed using an AR2000 rheometer (TA Instruments, New Castle Del.). Hydrogel kinetics were tested using a flow procedure in which 900 μl of hydrogel was dispensed on the rheometer surface under a 2°, 40 mm diameter aluminum cone. The viscosity of each hydrogel was over a range of applied shear stresses from 0 to 60 Pa at constant temperature (25° C.). To analyze the changes in each hydrogel over time, an oscillation test was performed on each of the six hydrogel compositions. 900 μl of hydrogel was added to the platform of the rheometer below the aluminum cone. A time sweep of 20 minutes was carried out with the frequency of the oscillation held at 1 Hz at constant 1% strain. After 90 seconds, a stoichiometric quantity of hydrogen peroxide was added around the edge of the aluminum cone in three equal amounts following the equation: Total Hydrogen Peroxide Amount (μl)=(0.496)(X)(Y); wherein X=Percentage of Tyramine base substitution (i.e., 1%=1) and Y=Concentration of Hydrogel (i.e., 5 mg/ml=5).

As the reaction proceeded, the shear moduli, G′ and G″, as well as the delta phase difference between them, was quantified. As a control, an oscillation test was performed in which no peroxide was added to the hydrogel to ensure that changes to the hydrogel were due to the addition of the peroxide agent. The complex shear modulus for each hydrogel was acquired using the oscillation data by Equation 1 below:

G*=\sqrt{G′²+G″²} [1]

### Example 2

### Using a Fenestrated Needle to Form Axially Homogeneous Implants

Turning to FIG. 3, one challenge encountered during the testing of BMPs and flowable matrix materials was the uneven mixing of these materials during implant formation, which resulted in relatively uneven implants that might be more prone to mechanical failure and/or inconsistent biological activity due to uneven concentrations of BMP and/or granules within the matrix. One means of improving the homogeneity of the implants was the use of a fenestrated needle (FIG. 3A) that included multiple ports along its length through which solutions could be flowed into a chamber (such as a syringe barrel) packed with granules. In the example shown in FIG. 3, a fenestrated needle with a closed distal tip is inserted more or less into the center of an elongated chamber such as a syringe barrel that was at least partially filled with granules. Thereafter, a solution of fluorescently-tagged BMP-2 and hydrogen peroxide (a reagent required for crosslinking the biocompatible matrix) was flowed into the chamber through the needle. Following the BMP-2/hydrogen peroxide solution, a solution of functionalized hyaluronic acid was flowed through the needle and over the granules. Alternatively, the solutions were sequentially applied to the granules through the luer tip of the syringe without a centrally-positioned fenestrated needle. In both cases, fluid flowed in a proximal-to-distal direction. In the absence of the fenestrated needle, solutions flowing into the chamber necessarily contacted granules located proximally prior to reaching distally located granules. In contrast, the fenestrated needle permitted near-simultaneous contact of fresh solution with granules throughout the long axis of the chamber. As is illustrated in FIGS. 3C through 3N, the use of a fenestrated needle resulted in substantially more uniform distribution of BMP (fluorescent signal) along both radial (e.g. from center to edge), and axial (proximal to distal) dimensions of the resulting implant when compared to the distribution achieved with a comparable bolus application of solutions of BMP/hydrogen peroxide and biocompatible matrix.

### Example 3

### Using a Static Mixer to Form Homogenous Implants

To create implants comprising BMP-loaded granules within a biocompatible matrix, implant components were placed into two syringes and passed back and forth through the static mixer connector. Hydrogel material was added to one syringe, while the granules, desired protein (or dye) and 0.09% hydrogen peroxide were combined in the other syringe. The mixing connector was then threaded tightly onto the horizontally held syringes so that none of the components could leave the system and/or prematurely mix. To mix the hydrogel and granule components, the hydrogel syringe was plunged first so that the hydrogel moved into the syringe containing the granule mixture. The granule mixture syringe was then plunged so that all of the components moved through the static mixer into the other syringe. This mixing was done 10 times over a period of 5 seconds. During this process, the device was rotated along its axis to mitigate settling of the granules. After the 5 second mixing time, the device was set vertically so all materials flowed into the bottom of the syringe and the implant set up. The shape of the syringe and the amount of material used (1800 μl) formed a 20 mm long cylindrical implant with a diameter of 10 mm.

### Example 4

### Mechanical Implant Analysis

Parallel plate rheometry was used to evaluate the mechanical properties of implants produced using the static mixing device. The AR2000 rheometer was used to perform dynamic rheological tests. Implants were created using the single crossbar design to mix 3% tyramine substitution at 10 mg/ml concentration, Trypan Blue dye, 0.09% peroxide and granules. (Table 1).

TABLE 1
SCAFFOLD MIXTURE CONCENTRATIONS

| Formulation | Granules | Buffer | Hydrogel | Peroxide | Other |
|---|---|---|---|---|---|
| 0% Granules | No | 200 μL PBS | 1500 μL | 25 μL | 15 μL Trypan |
| by volume | granules | (diluted with | hydrogel | H₂O₂ | Blue |
| | | albumin 1:250 | | | (optional) |
| | | or BMP 1:1200) | | | |
| 20% Granules | 0.18 g | 360 μL PBS | 1440 μL | 24 μL | 18 μL Trypan |
| by volume | granules | (diluted with | hydrogel | H₂O₂ | Blue |
| | | albumin 1:250 | | | (optional) |
| | | or BMP 1-1200) | | | |
| 25% Granules | 0.225 g | 450 μL PBS | 1350 μL | 22.5 μL | 18 μL Trypan |
| by volume | granules | (diluted with | hydrogel | H₂O₂ | Blue |
| | | albumin 1:250 | | | (optional) |
| | | or BMP 1-1200) | | | |
| 30% Granules | 0.27 g | 540 μL PBS | 1260 μL | 21 μL | 18 μL Trypan |
| by volume | granules | (diluted with | hydrogel | H₂O₂ | Blue |
| | | albumin 1:250 | | | (optional) |
| | | or BMP 1-1200) | | | |

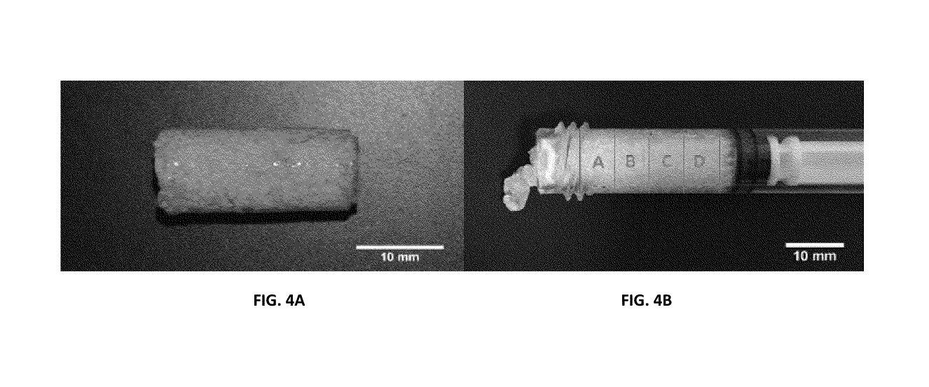

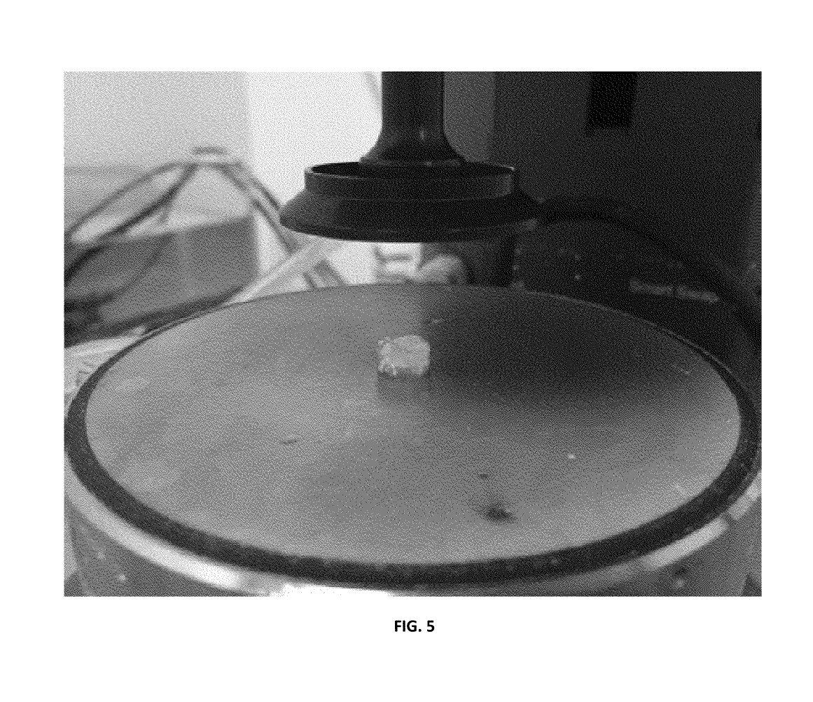

The resulting implants (10 mm diameter, 20 mm length) were cut into four 5 mm thick sections (FIG. 4A-B). These sections were labeled from A, corresponding to the section closest to the opening of the syringe when cut, to D, the section farthest from the opening of the syringe. Sections were stored in 100 μl of phosphate buffered saline (PBS) to prevent drying out. The mass of each slice was measured, and the density calculated by dividing the mass by the 0.393 ml volume. A 40 mm diameter aluminum parallel plate configuration was used to apply a compressive force on each implant disc (FIG. 5). The rheometer plate was moved to 100 μm above the top of the sample and lowered at a constant rate (10 μm/s) as it compressed the sample to 50% strain (2.5 mm). The compression force was recorded as a function of the height of the rheometer plate. Based on these values, the true stress and true strain curves for each sample was calculated. The elastic modulus was calculated based on the linear region of this graph and compared across samples. These tests were completed with granule concentrations of 20%, 25% and 30% by volume. Control tests included testing hydrogel with no granules, and a 30% granule concentration mixed using a hollow tube connector with no mixing geometries.

### Example 5

### Fluorescence Plate Reader Analysis

Implants were created using fluorescently tagged albumin Alexa Fluor-647 (AF647) protein (647 nm excitation; 670 nm emission wavelength). The single crossbar prototype was used to mix 3% tyramine substitution at 10 mg/ml concentration, tagged albumin AF647 diluted 1:250 in PBS, 0.09% peroxide and granules (Table 1). After the mold was cut into 5 mm sections and mechanical testing data was collected, each section was placed into a single well of a 48 well plate with 100 μl PBS. The plate was read with a SpectraMax™ M5 microplate reader (Molecular Devices, LLC, Sunnyvale, Calif.) to determine the intensity of fluorescence within each 5 mm slice. The florescence intensities were then normalized with the amount of albumin protein within each section. Following preliminary testing with albumin, Alexa Fluor-488 (AF488) tagged BMP-2 (488 nm excitation wavelength; 520 nm emission wavelength) diluted 1:120 in BMP buffer (50 mM glutamic acid, 0.75% glycine, pH 3.75) was used to create the implant, and fluorescence was measured using the same approach.

### Example 6

### Hydrogel Setup and Stiffness Measurements

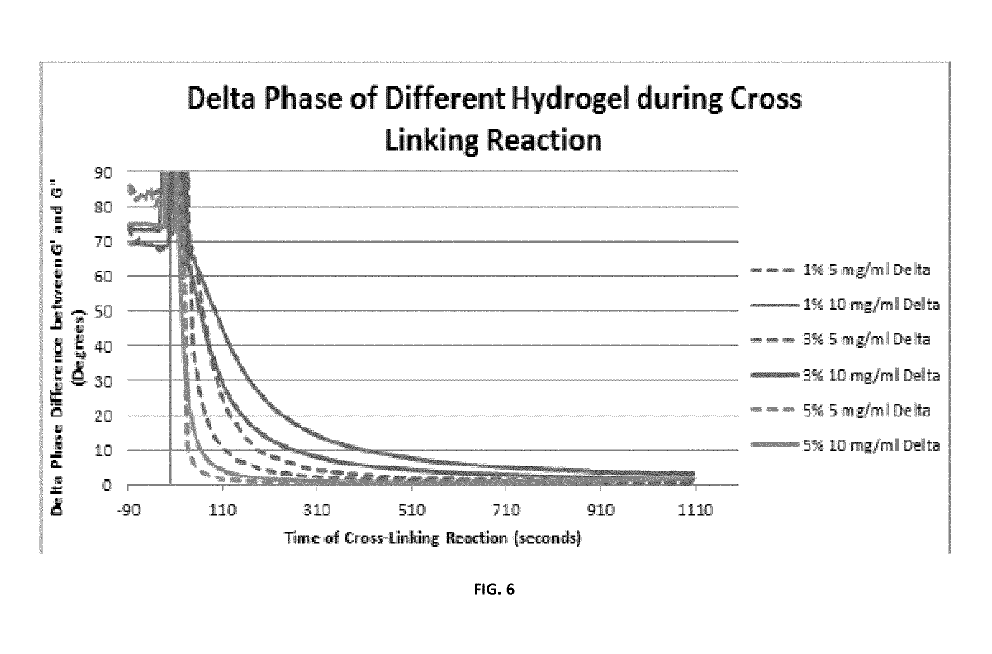

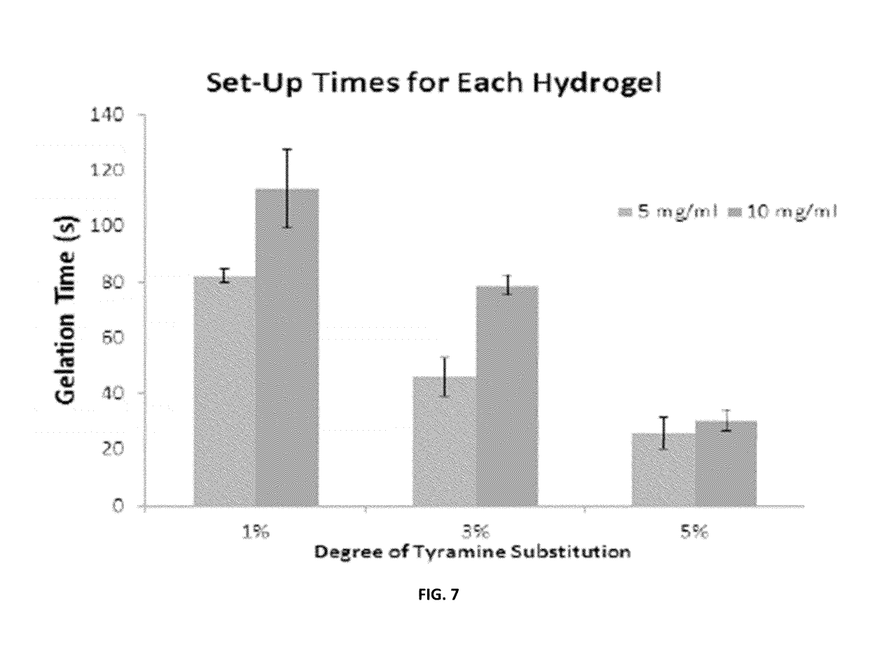

The relative peroxide-linking setup times and stiffness were characterized among various hydrogels (e.g., 1%, 3% and 5% tyramine base substitution; 5 mg/ml and 10 mg/ml concentrations). The steady decrease of each phase difference curve displays the progression of each hydrogel from a viscous liquid to an elastic solid (FIG. 6). The setup time was the time span from when the peroxide was added until the shear storage G′ (elastic) component of the shear modulus exceeded the shear loss G″ (viscous) component. Hydrogels with higher tyramine base substitutions and higher concentrations of the hydrogel set up slower within a given tyramine base substitution (FIG. 7). The hydrogels with the longest setup times were 1% substitution at 5 mg/ml, 1% at 10 mg/ml and 3% substitution at 10 mg/ml (Table 2).

TABLE 2
Times for Completion of Cross-Linking Reactions

| | Hydrogel Concentration | | | | | |
|---|---|---|---|---|---|---|
| | 1%, | 1%, | 3% | 3% | 5% | 5% |
| | 5 mg/mL | 10 mg/mL | 5 mg/ml | 10 mg/mL | 5 mg/mL | 10 mg/mL |
| Cross-Linking Completion | 82.4 ± 2.4 | 135.5 ± 14.3 | 45.9 ± 6.7 | 79.0 ± 3.6 | 25.9 ± 5.7 | 30.3 ± 3.8 |
| Time (S ± S.E.) | | | | | | |

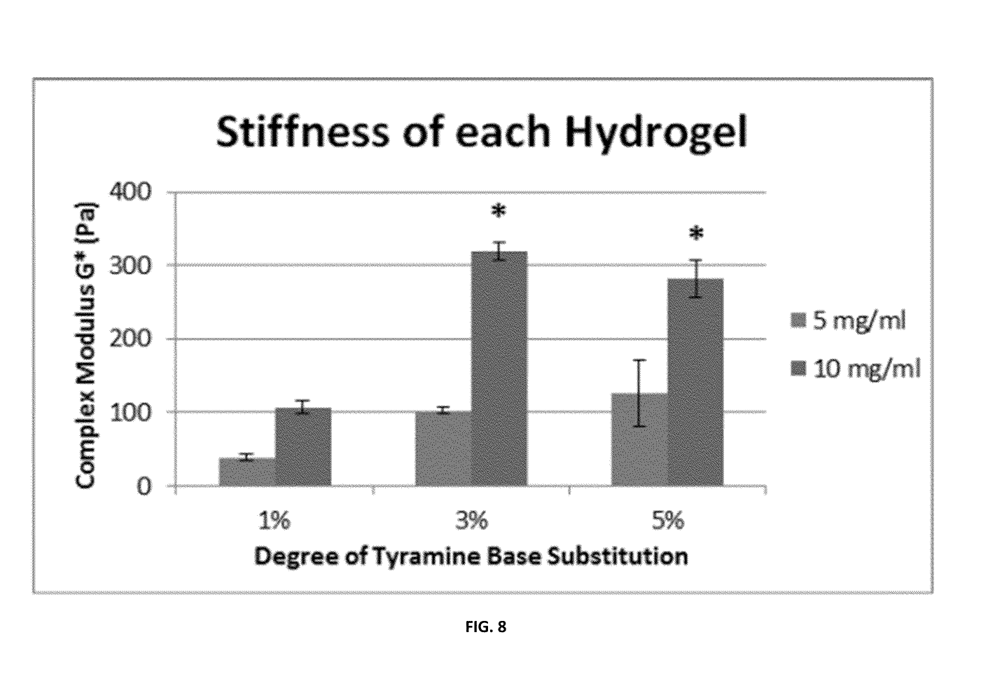

The complex shear modulus G* determined the relative stiffness among the tested hydrogels. Hydrogels with higher concentrations had greater complex moduli within a given tyramine base substitution (FIG. 8). The hydrogels with the stiffest final setup were 3% substitution at 10 mg/ml and 5% substitution at 10 mg/ml (FIG. 8; Table 3). The hydrogel with 3% substitution at 10 mg/ml was chosen.

TABLE 3
Maximum Stiffness Values Measured During 20-minute Cross-Linking Reactions

| | Hydrogel Concentration | | | | | |
|---|---|---|---|---|---|---|
| | 1%, | 1%, | 3% | 3% | 5% | 5% |
| | 5 mg/mL | 10 mg/mL | 5 mg/ml | 10 mg/mL | 5 mg/mL | 10 mg/mL |
| G* Complex Shear | 38.8 ± 4.2 | 107.5 ± 8.5 | 103.0 ± 4.2 | 318.8 ± 11.7 | 126.7 ± 45.0 | 281.7 ± 25.8 |
| Modulus (Pa ± S.E.) | | | | | | |

### Example 7

### Device Prototyping

Following hydrogel selection, a device was developed for uniformly mixing the implant components. Multiple concepts for mixing devices were created to begin the design process. These concepts included, but are in no way limited to, a double barrel syringe, a rotating blade mixer, a rolling tube method and a static mixer. Due to the quick setup time of the hydrogel crosslinking reaction, standard mixing with a stir bar would not provide adequate distribution of granules before the gel would shear from mixing. The static mixer was chosen because it best met the functional design requirements. The static mixer is simple to operate, inexpensive to manufacture, disposable and mixed the components of the implant uniformly while minimizing waste. Additionally, the device is able to be used with commercial syringes, eliminating the time and cost associated with manufacturing new syringes.

The static mixer design went through several iterations to create a connector that provided that shortest distance between syringes, as well as an airtight fit. Previous versions of the mixer were too long, causing material to get caught inside of the connector, and were not airtight, allowing material to seep out of the device. Both machined and 3D printed versions of the device were short and airtight, but the machined version was more difficult and time consuming to reproduce. The 3D printed mixers were redesigned to include a variety of internal geometries configured to disrupt the flow of material through the mixer so that granules would be evenly dispersed throughout the hydrogel. Some mixers, such as the double crossbar and semi-sphere designs, did not allow all of the material to flow through, leaving wasted material in the connector. The single crossbar design was selected, in part, because it provided even mixing while allowing the majority of the material to flow through.

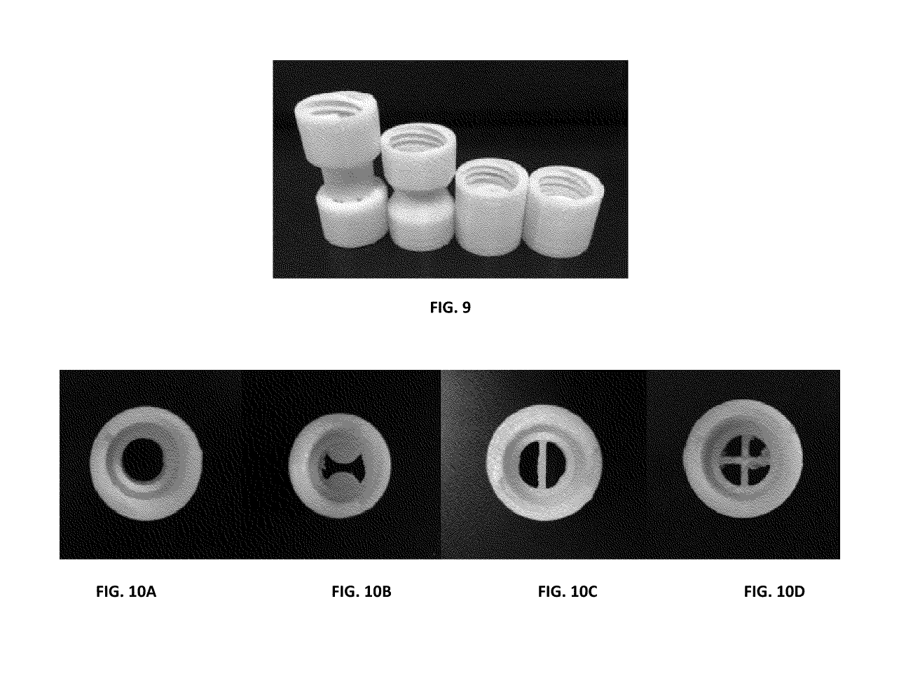

The static mixer design was selected from four initial concepts. 3D-printed prototypes were designed to fit the screw thread of existing syringes to make them airtight. Initially, rubber O-rings were added to the inside of the mixer to create an airtight seal. Further design alterations led to better fitting threads, eliminating the need to O-rings. To minimize waste the distance between the ends of the syringes when connected to the mixer was reduced. Progression of the mixer shape is shown in FIG. 9. As shown in FIGS. 10A-D, once the shape and threads of the design were finalized, variations of the internal mixing structure, such as a hollow tube (10A), semi-spheres (10B), single crossbar (10C) and double crossbar (10D), were created to test their mixing ability.

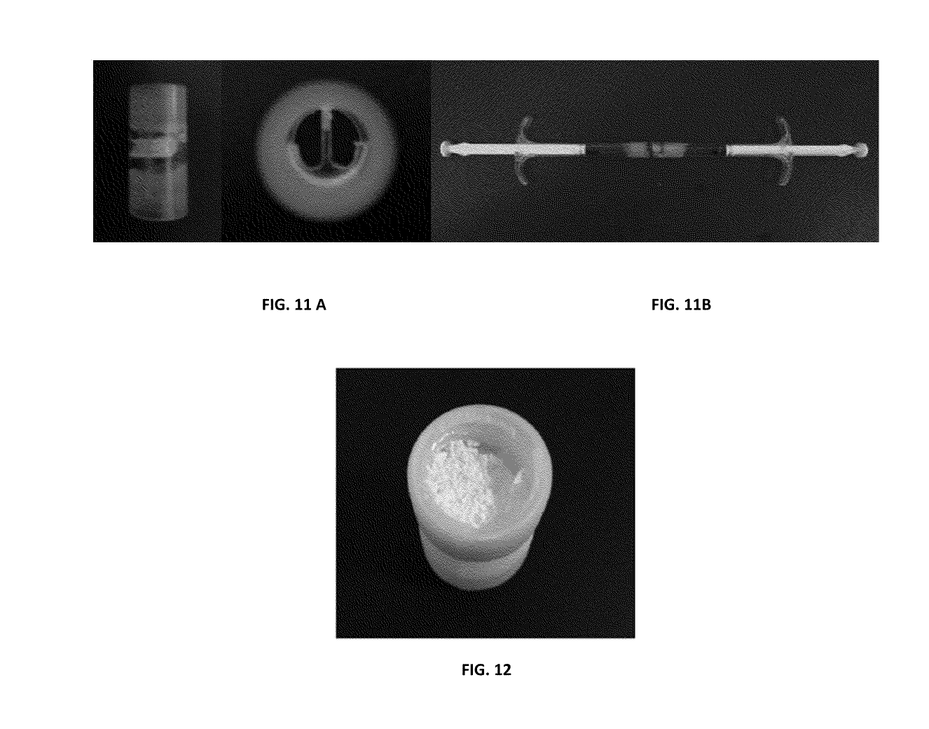

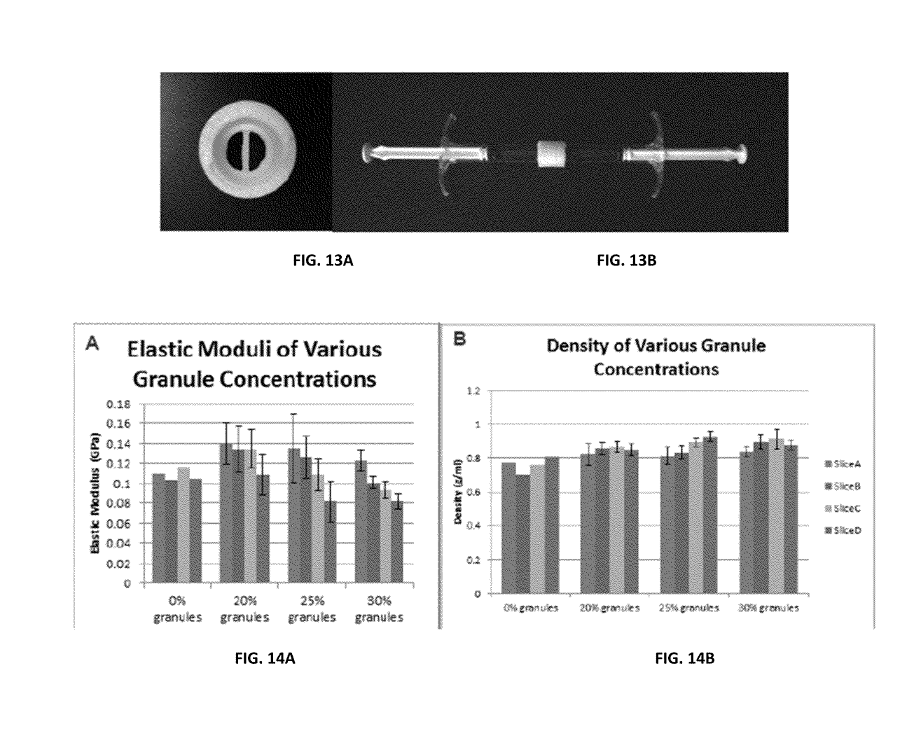

Due to the temperature limitations of the 3D-printer, transparent plastic for viewing mixing within the mixing device could not be used. Therefore, additional prototypes of the static mixers were created by milling clear plastic tubing to have a press fit seal with the commercial syringes without threads (FIGS. 11A-B). Although these parts allow the mixing procedure to be viewed through the device, machining restrictions limited the variability in internal geometries. 3D-printed prototypes proved much more reproducible and time efficient to produce and modify. The different mixing geometries were initially evaluated by visually comparing the implants and waste they produced. A relatively large amount of waste material remained in the static mixer component of the double crossbar and semi-sphere prototypes (FIG. 12). Therefore, the single crossbar 3D-printed design was chosen as the final device design (FIG. 13).

### Example 8

### Assessment of Uniformity of Produced Construct

Once the mixer design was finalized, the produced scaffolds were tested for uniformity and reproducibility. Uniform mechanical strength of the scaffold ensures even bone growth during recovery. If the mechanical properties of the scaffold are non-uniform, the developing bone may also vary in strength and density. Thus, formed implants were tested for their density and elastic modulus across four slices. Results demonstrate that the density across slices within a given scaffold was relatively uniform, with a deviation of approximately 4%. Additionally, the scaffolds for a given granule concentration were reproducible, as each test of a single composition exhibited relatively equal densities, with a variance of approximately 5%. Therefore, the mixing device is able to repeatedly create uniform scaffolds in relation to their density distribution and meets he requirement of being within a 10% variance. Some of the variance that did occur during these tests may be attributed to imperfectly sized slices, since the gel is flexible and could warp while being cut into sections.

This consistency was not seen when analyzing the elastic modulus of the scaffolds. The 30% granule constructs resulted in a more uniform distribution, deviating by only approximately 9% across slices, compared to the approximately 11% deviation seen in the other granule concentrations. While this difference between slices for the varying scaffolds is only approximately 2%, it was sufficient to distinguish the 30% concentration as the only one meeting the 10% variance requirement for uniformity between slices.

The 30% granule implants had a deviation between implants of approximately 8%, meeting the 10% variance requirement for reproducibility, while the 20% and 25% granule implants did not. Scaffolds tended to have a relatively lower elastic modulus in the proximal slice (A) than at the distal end (D). This could be attributed to the falling of the heavier granules in the hydrogel at the end of mixing while the scaffold is completing its cross-linking. Despite this factor, the 30% granule implants satisfy the 10% variance requirement for both uniformity and reproducibility.

Another measure of uniformity is the spread of granules and protein throughout the length of the implant. The fluorescence intensity from a portion of the implant indicates the amount of protein present. This also indirectly indicates the presence of granules because the fluorescent protein localizes around them. The 30% granule concentration proved to be the only composition that had less than a 10% deviation in uniformity of fluorescence intensity, further confirming that the 30% granules by volume was the desired concentration. A control test using a hollow tube as the mixing device was completed using 30% granules to validate the mixing due to the selected internal geometry. Two control tests resulted in an approximately 12% deviation of fluorescence intensity throughout the construct. This did not satisfy the 10% error threshold and supports the efficacy of the single crossbar prototype as the optimal design. The 0% granule control construct concentration exhibited a significant difference in fluorescence emission intensity as compared to equivalent slices from other compositions. This indicates that the granules play a significant role in albumin protein localization and consolidate the protein into smaller areas. Although this result was not re-tested with BMP-2 due to limited material and time, albumin serves as a satisfactory substitute due to its low cost, biocompatibility and similar calcium phosphate binding properties. Although the albumin was only tested twice, preliminary results of plate reader testing with BMP-2 showed that 20% and 30% granule concentrations corresponded with the albumin results.

Development of the mixing device and method of its use required determining the optimal scaffold composition. The functional specifications required of the mixing device were to produce implants with uniform mechanical properties, dispersion of granules throughout the hydrogel and distribution of protein among the granules. Uniformity is defined as less than 10% variation between implants. After initial prototypes were developed, testing various granule concentrations identified the preferred range as 20-30% granules by volume. Lower granule concentrations provided too few granules for BMP binding and structural support. Concentrations exceeding 30% resulted in inefficient hydrogel being available to bind the granules. Additionally, the hydrogel volume between granules permits osteogenic cells and blood vessels to more readily infiltrate the implant. When the implant was formed inside the syringe, the top portion tended to be misshapen. This layer was trimmed to allow the implant to have the desire shape and length. In the operating room this imperfectly shaped end will be cut off as the implant is shaped to fit the implantation site. These properties were tested via rheometer compression analysis, plate reader fluorescence testing and confocal microscopy imaging.

### Example 9

### Assessment of Intra-Implant Mechanical Properties

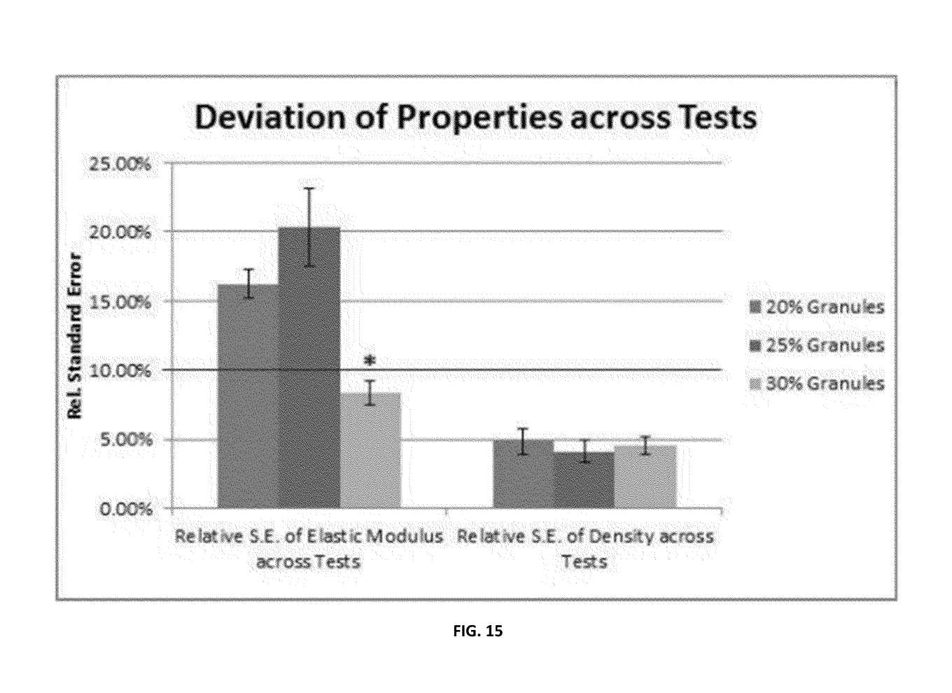

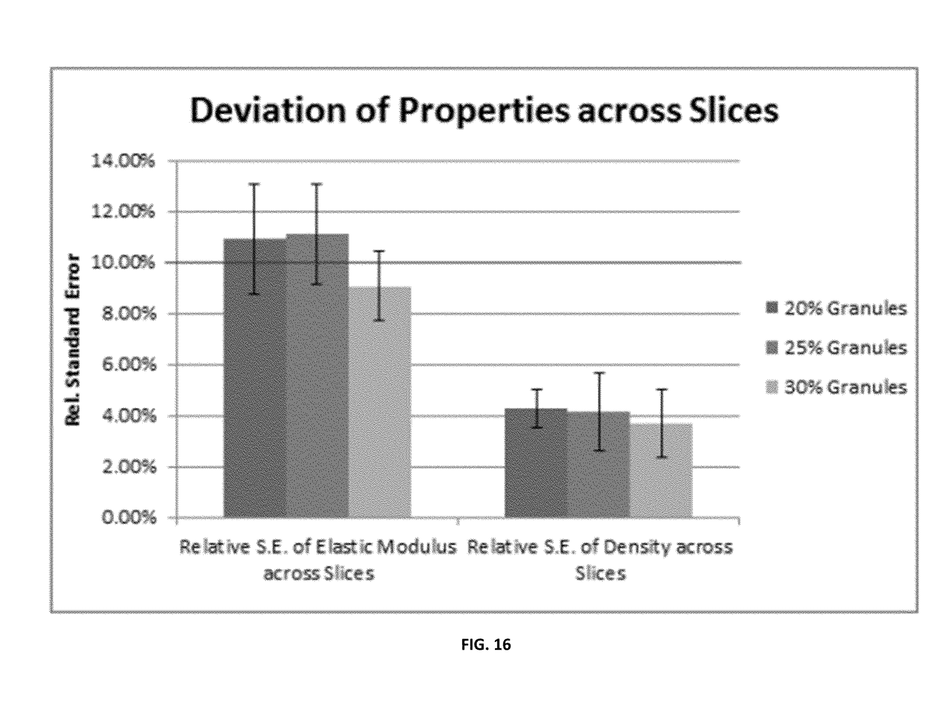

The elastic moduli along linear sections of produced constructs were calculated to determine the gradient of mechanical strength (FIG. 14A). Although the mechanical strength tended to decrease form the proximal (slice A) to distal (slice D) regions of the construct, the variance of the elastic moduli and density throughout the slices was minimal for the tested granule concentrations (FIG. 14B). The deviations between whole constructs of the same granule concentration were compared to determine reproducibility. Constructs composed of 30% granules produced statistically lower elastic moduli deviation across tests compared to constructs created with 20% and 25% granules. Relative standard errors of approximately 5% in density across tests were found in produced scaffolds containing 20%, 25% and 30% granule concentrations (FIG. 15). The deviations between slices within constructs of the same granule concentration were compared to determine uniformity. No significant difference was determined between constructs for the elastic moduli and density deviations across slices. All three density deviations were below the 10% error threshold defined for uniformity; however only the 30% granule concentration was below the threshold for the elastic modulus deviation (FIG. 16).

### Example 10

### Assessment of Implant Protein Distribution

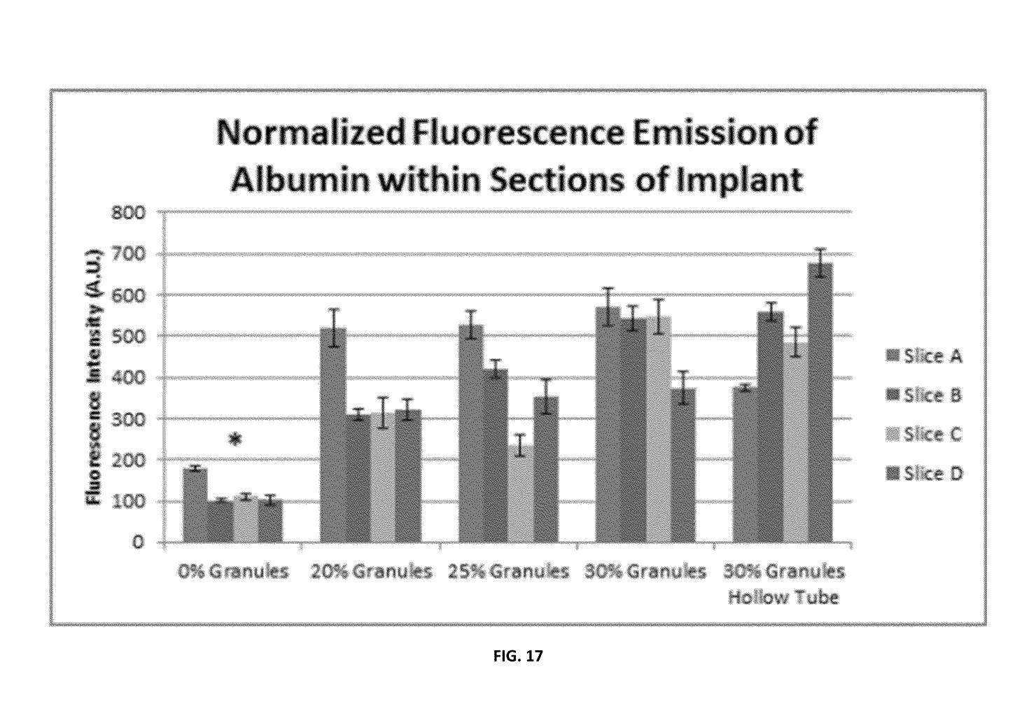

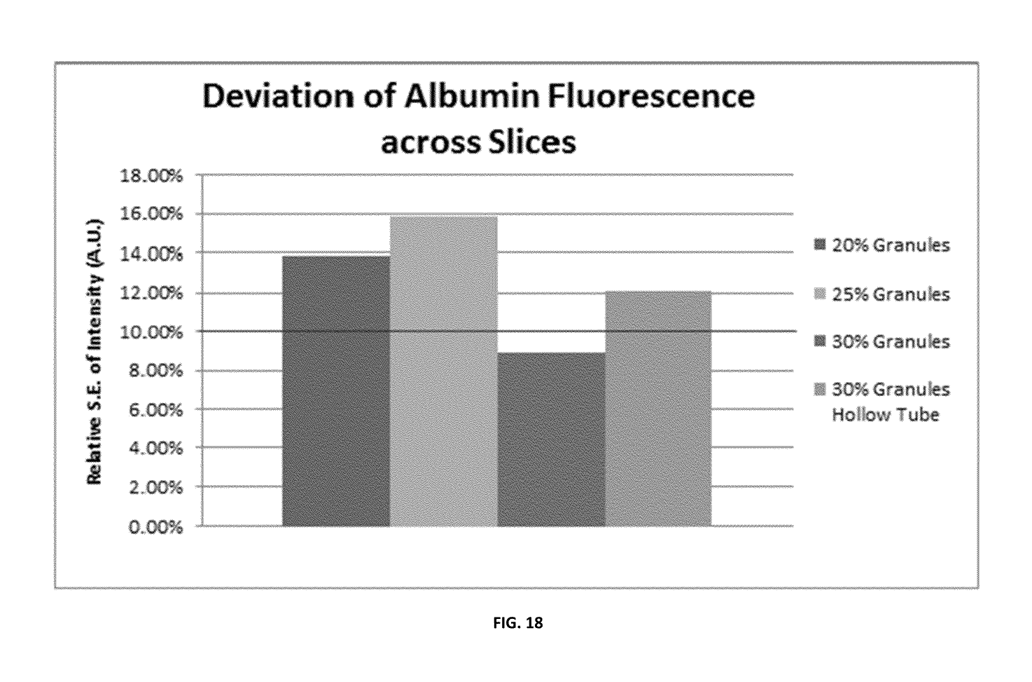

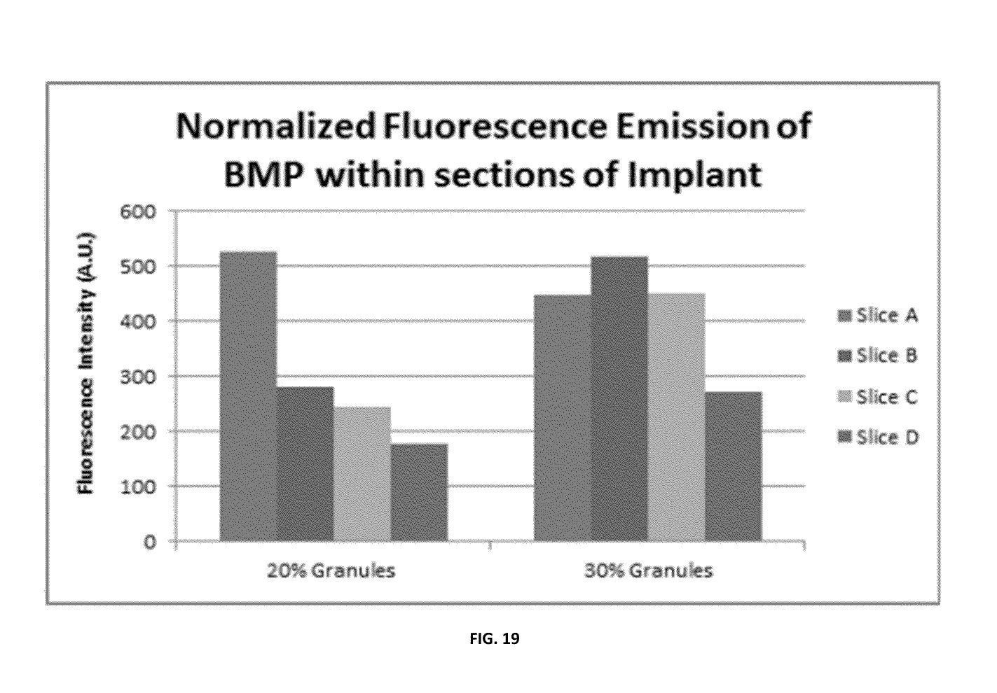

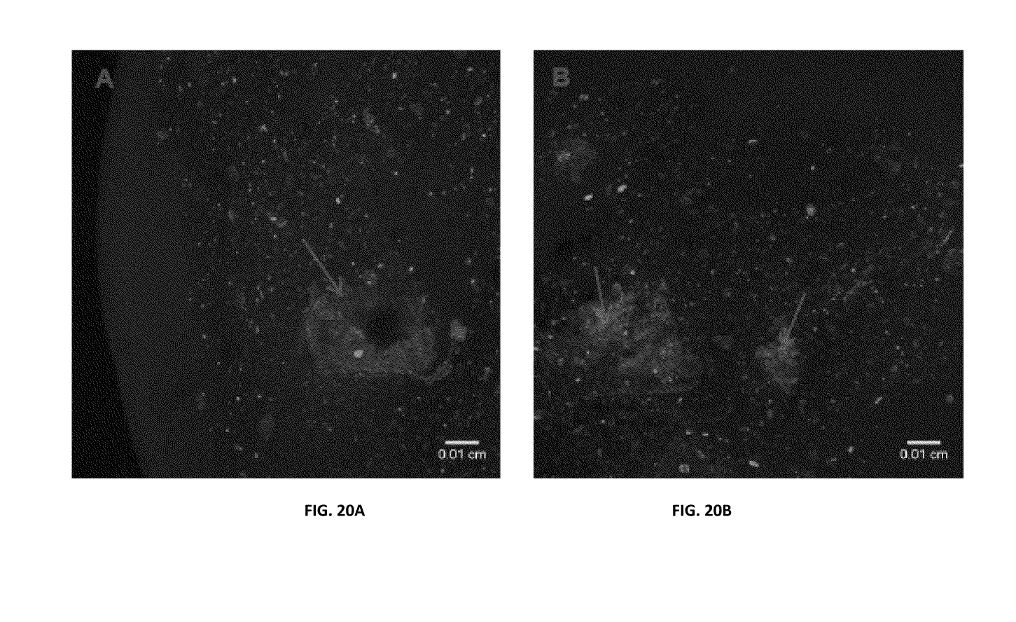

The spread of the fluorescence emission showed that constructs produced with the static bar mixing device did not have statistically significant differences in fluorescent emissions among their slices (FIG. 17). The 0% granule composition was shown to have statistically lower fluorescent emission intensity compared to the other compositions. The average difference in fluorescence for the constructs containing 30% granules was approximately 90%, while the 20% and 25% implants were approximately 14% and 16%, respectively (FIG. 18). Although the BMP-2 fluorescence reading was completed only once for the 20% and 30% granule concentrations, the fluorescence measurements were similar to the albumin values of their respective concentrations (FIG. 19).

### Example 11

### Assessment of Implant Protein Content Using Confocal Microscopy

Confocal microscopy was used to verify the results of the plate reader fluorescence data. Implants were created using the single crossbar device prototype to mix 3% tyramine substitution at 10 mg/ml hydrogel concentration, AF488 tagged BMP-2 diluted 1:120 in BMP buffer, 0.09% peroxide and 30% granules by volume. After cutting the construct into 5 mm sections and performing mechanical testing, each section was cut down to 1 mm and fixed to a glass slide with an elevated slide cover and viewed under the confocal microscope. A stack of 10 images spanning 100 microns were collected from the center and edge of each section and analyzed using ImageJ image processing software. The average fluorescent intensities of the collapsed stack for the granule and hydrogel areas in each image were collected.

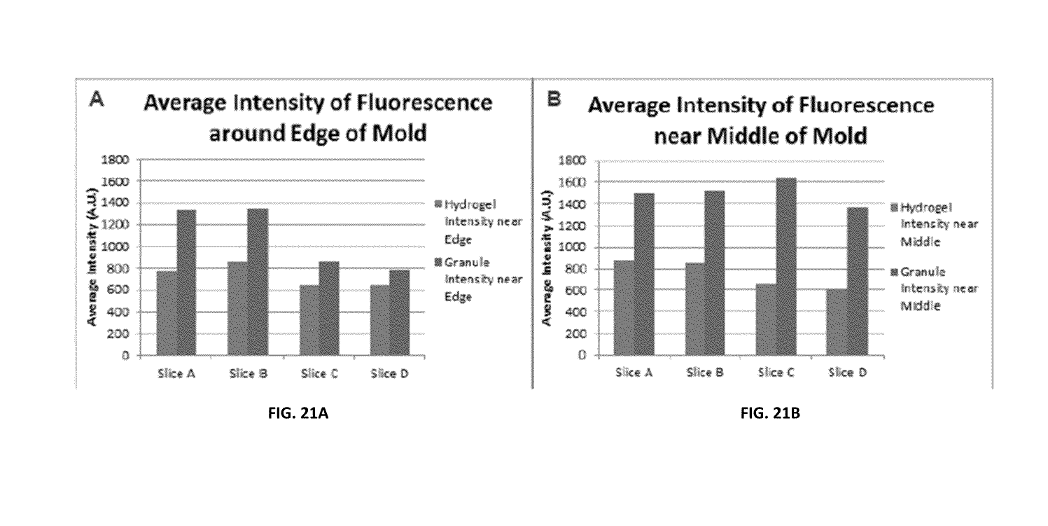

The maximum intensity collapsed stack images for confocal microscopy of BMP-2 is shown in FIGS. 20A-B. The protein is shown to be concentrated around the granules (see arrows). Areas with less concentrated fluorescence are regions of hydrogel. This was confirmed by average fluorescence intensity values comparing the hydrogel and granule regions at the middle and edge of each slice (FIGS. 21A-B).

As expected, images showed the presence of protein in each slice specifically localized around each granule. Fluorescence intensity was obtained by calculating the average intensity within regions of granule and regions of hydrogel in each image. Although this procedure was only performed for a single implant, the protein intensity was much higher in the granules than in the hydrogel. However, there was a higher variability in fluorescence intensity measurements at the edges of the slices as compared to measurements closer to the middle.

An unexpected side effect seen in the confocal images was clouds of calcium phosphate dust surrounding each granule. There are clear portions of each image that contain small pieces of granule debris bound with the protein. This effect could result from granules being broken during the mixing process, or a side effect of long term storage. Further research is required to determine why this debris is present and what effect, if any it will have on the performance of the implant in vivo.

As disclosed herein, the properties of the hydrogel used greatly influenced the ability of the mixing device to meet the functional requirements of the synthetic bone graft material. Once the peroxide cross-linking reaction has completed, the components of the implant can no longer be mixed due to the risk of shearing. Mixing potential increases with prolonged mixing time, so a longer setup time correlates with a more uniform distribution with a produced implant. The setup time identified through the hydrogel characterization experiments provided relative gelation speeds among the tested hydrogels. Hydrogels with a higher tyramine base substitution tended to have a faster setup time, and hydrogels at a higher concentration set up slower relative to others within a substitution percentage. Although the setup time when mixing the construct components was significantly faster under device mixing conditions, the relative reaction times among tested hydrogels was determined by the characterization procedures. Therefore, hydrogels with the longest relative setup times (1% substitution at 5 mg/ml, 1% substitution at 10 mg/ml and 3% substitution at 10 mg/ml) were more amenable to use with the mixing device than those that setup more quickly (5% substitution at 5 mg/ml and 5% substitution at 10 mg/ml). Additionally, the shear modulus of each hydrogel was determined as an indicator of mechanical stability. To transition to a clinical setting, the implant produced by the mixing device must withstand forces in the body. Therefore, hydrogels with a significantly greater stiffness (3% substitution at 10 mg/ml and 5% substitution at 10 mg/ml) are a better fit for use with the mixing device. The hydrogel with 3% tyramine base substitution at a concentration of 10 mg/ml was best suited as a synthetic bone graft material due to the relatively longer mixing time and higher stiffness needed for clinical use.

### CONCLUSION

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “consists essentially of” means excluding other materials that contribute to function, unless otherwise defined herein. Nonetheless, such other materials may be present, collectively or individually, in trace amounts.

As used in this specification, the term “substantially” or “approximately” means plus or minus 10% (e.g., by weight or by volume), and in some embodiments, plus or minus 5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

Certain embodiments of the present invention have described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

What is claimed is:

1. A method of making an osteoinductive scaffold, the method comprising:
contacting a calcium ceramic granule with a solution comprising an osteoinductive material, thereby associating the osteoinductive material with an interior surface of the ceramic granule; and
placing the granule within a biocompatible matrix.

2. The method of claim 1, wherein the step of placing the granule within the biocompatible matrix includes contacting the granule with a biocompatible matrix material and reacting the matrix material by at least one of polymerizing the matrix material and cross-linking the matrix material, thereby forming the biocompatible matrix.

3. The method of claim 1, wherein the biocompatible matrix is selected from the group consisting of hyaluronic acid (HA), modified HA, collagen, gelatin, fibrin, chitosan, alginate, agarose, a self-assembling peptide, whole blood, platelet-rich plasma, bone marrow aspirate, polyethylene glycol (PEG), a derivative of PEG, poly(lactide-co-glycolide), poly(caprolactone), poly(lactic acid), poly(glycolic acid), a poloxamer, and copolymers thereof.

4. The method of claim 1, wherein the granule includes a material selected from the group comprising monocalcium phosphate monohydrate, dicalcium phosphate, dicalcium phosphate dehydrate, octocalcium phosphate, precipitated hydroxyapatite, precipitated amorphous calcium phosphate, monocalcium phosphate, alpha-tricalcium phosphate (α-TCP), beta-tricalcium phosphate (β-TCP), sintered hydroxyapatite, oxyapatite, tetracalcium phosphate, hydroxyapatite, calcium-deficient hydroxyapatite, and combinations thereof.

5. The method of claim 1, wherein the osteoinductive material is selected from the group consisting of bone morphogenetic protein 2 (BMP-2), BMP-3, BMP-4, BMP-5, BMP-6, BMP-7, BMP-9, a designer BMP, fibroblast growth factor, insulin-like growth factor, platelet-derived growth factor, transforming growth factor beta (TGF-β), and combinations thereof.

6. A method of treating a patient, comprising the steps of:
associating a ceramic granule with an osteoinductive material;
associating the ceramic granule with a biocompatible matrix material, thereby forming an implant; and
placing the implant within or adjacent to a bone of the patient.

7. The method of claim 6, wherein the step of associating the ceramic granule with the biocompatible matrix material includes contacting the granule with a matrix material and reacting the matrix material by at least one of polymerizing the matrix material and cross-linking the matrix material, thereby forming the biocompatible matrix.

8. The method of claim 7, wherein the step of reacting the biocompatible matrix material to form the gelled biocompatible matrix takes between 30 seconds and 5 minutes, and the step of associating the ceramic granule with the biocompatible matrix material includes at least one of mixing the granule and the biocompatible matrix material and flowing the biocompatible matrix material over the granule.

9. The method of claim 6, wherein the biocompatible matrix material is selected from the group consisting of hyaluronic acid (HA), modified HA, collagen, gelatin, fibrin, chitosan, alginate, agarose, a self-assembling peptide, whole blood, platelet-rich plasma, bone marrow aspirate, polyethylene glycol (PEG), a derivative of PEG, poly(lactide-co-glycolide), poly(caprolactone), poly(lactic acid), poly(glycolic acid), a poloxamer, and copolymers thereof.

10. The method of claim 6, wherein the granule includes a material selected from the group comprising monocalcium phosphate monohydrate, dicalcium phosphate, dicalcium phosphate dehydrate, octocalcium phosphate, precipitated hydroxyapatite, precipitated amorphous calcium phosphate, monocalcium phosphate, alpha-tricalcium phosphate (α-TCP), beta-tricalcium phosphate (β-TCP), sintered hydroxyapatite, oxyapatite, tetracalcium phosphate, hydroxyapatite, calcium-deficient hydroxyapatite, and combinations thereof.

11. The method of claim 6, wherein the osteoinductive material is selected from the group consisting of bone morphogenetic protein 2 (BMP-2), BMP-3, BMP-4, BMP-5, BMP-6, BMP-7, BMP-9, a designer BMP, fibroblast growth factor, insulin-like growth factor, platelet-derived growth factor, transforming growth factor beta (TGF-β), and combinations thereof.

12. The method of claim 6, further comprising a step of contacting the biocompatible matrix material with a porogen.

13. The method of claim 6, wherein a concentration of the osteoinductive material and one or more of the granule and the biocompatible matrix material is substantially the same at opposite first and second ends of the implant.

14. A kit for treating a patient, comprising:
a first vessel containing a plurality of calcium ceramic granules;
a second vessel configured to fluidly couple to the first vessel, the second vessel containing a first solution; and
a third vessel configured to fluidly couple to the first vessel, the third vessel containing a second solution comprising a material configured to form a biocompatible matrix.

15. The kit of claim 14, further comprising:
an instruction set comprising a method of treating a patient, the method comprising the steps of;
flowing the first solution into the first vessel, thereby associating the ceramic granules with the osteoinductive material; and
flowing the second solution over the plurality of ceramic granules, thereby embedding at least one of the plurality of ceramic granules in a biocompatible matrix.

16. The kit of claim 15, wherein the step of flowing the second solution over the plurality of ceramic granules includes reacting a material in the second solution by at least one of polymerizing the material and cross-linking the material, thereby forming the biocompatible matrix.

17. The kit of claim 15, wherein the biocompatible matrix is selected from the group consisting of hyaluronic acid (HA), modified HA, collagen, gelatin, fibrin, chitosan, alginate, agarose, a self-assembling peptide, whole blood, platelet-rich plasma, bone marrow aspirate, polyethylene glycol (PEG), a derivative of PEG, poly(lactide-co-glycolide), poly(caprolactone), poly(lactic acid), poly(glycolic acid), a poloxamer, and copolymers thereof.

18. The kit of claim 14, wherein the granule includes a material selected from the group comprising monocalcium phosphate monohydrate, dicalcium phosphate, dicalcium phosphate dehydrate, octocalcium phosphate, precipitated hydroxyapatite, precipitated amorphous calcium phosphate, monocalcium phosphate, alpha-tricalcium phosphate (α-TCP), beta-tricalcium phosphate (β-TCP), sintered hydroxyapatite, oxyapatite, tetracalcium phosphate, hydroxyapatite, calcium-deficient hydroxyapatite, and combinations thereof.

19. The kit of claim 14, wherein the osteoinductive material is selected from the group consisting of bone morphogenetic protein 2 (BMP-2), BMP-3, BMP-4, BMP-5, BMP-6, BMP-7, BMP-9, a designer BMP, fibroblast growth factor, insulin-like growth factor, platelet-derived growth factor, transforming growth factor beta (TGF-β), and combinations thereof.

20. The kit of claim 14, further comprising at least one of a static mixing element disposable between the first vessel and at least one of the second and third vessels and a fenestrated needle disposable within the first vessel and fluidly connectable to at least one of the second and third vessels.
